(19) United States
(12) Patent Application Publication
(10) Pub. No.: US 2004/0088748 A1
(43) Pub. Date: May 6, 2004

(54) Method of performing transgenesis

(76) Inventor: Anthony C.F. Perry, Chuo-ku (JP)

Correspondence Address: HOFFMANN & BARON, LLP, 6900 JERICHO TURNPIKE, SYOSSET, NY, 11791, US

(21) Appl. No.: 10/422,054

(22) Filed: Apr. 23, 2003

Related U.S. Application Data
(63) Continuation of application No. 10/285,818, filed on Oct. 31, 2002, now abandoned.
Application No. 10/285,818 is a continuation of application No. 09/371,648, filed on Aug. 10, 1999, now Pat. No. 6,376,743.
(63) Continuation of application No. 09/762,924, filed on Apr. 23, 2001.
Application No. 09/762,924 is a 371 of international application No. PCT/US99/18429, filed on Aug. 11, 1999.
(60) Provisional application No. 60/133,970, filed on May 13, 1999.
(60) Provisional application No. 60/134,251, filed on May 13, 1999.

(51) Int. Cl. A01K 67/027

(52) U.S. Cl. 800/21; 119/300

(57) ABSTRACT

The invention provides a method for generating transgenic animals and cells by the coinsertion of nucleic acid and a nucleus into an unfertilized oocyte. Preferably, the coinsertion is by microinjection and more preferably by piezo-electrically actuated microinjection. Transgene (tg) expressing embryos are here produced following coinjection of unfertilized mouse oocytes with sperm heads and exogenous DNA encoding either a green fluorescent protein (GFP) or β-galactosidase reporter. The microinjected oocyte may be allowed to develop into differentiated cells or stem cells; into an embryo in vitro prior to transfer into a host surrogate mother, or it may be transferred directly into a host surrogate mother. Embryonic development can occur to term, such that the offspring possess transgenic modifications that may alter their characteristics (phenotype) and are, in turn, transmitted to their offspring.

The file of this patent contains at least one drawing executed in color. Copies of this patent with color drawing(s) will be provided by the United States Patent and Trademark Office upon request and payment of the necessary fee.

### BACKGROUND OF THE INVENTION

It is desirable to be able to modify the characteristics of whole animals and plants (or their embryonic precursors) in a prescribed manner. The method of choice for achieving this is known as transgenesis. “Transgenesis”, as used herein, is a process that results in the modification of genomes to carry newly-introduced DNA sequences. The process commonly entails the genomic integration of foreign, or transgene (tg), DNA sequences. The DNA sequences may encode desired characteristics, so that any transgenic animal (or plant) now carrying the genomic modification may possess one or more new characteristics endowed by it. Ideally, such genomic modifications are transmissible through the germline, such that they may be transmitted to offspring, and thereafter vertically along the lineage of the transgenic organism. It is often desirable that every cell in a given transgenic organism contains the tg; this is achievable via transgenesis. Transgenic animals and plants have great potential utility in agriculture, medicine and the production of bioactive compounds (neutraceuticals and pharmaceuticals). For example, the production of transgenic pigs expressing human major histocompatibility proteins on the surfaces of their cells would contribute to the usefulness of these animals in xenotrasnsplantation.

There presently exist several methods of transgenesis in animals. The first to become widely used—pronuclear microinjection—was developed in the mouse in the early 1980s and entails injection of tg DNA into one of the pronuclei of a one-cell embryo. In the first report of this method (Gordon, J. W., Scangos, G. A., Plotkin, D. J., Barbosa, J. A. & Ruddle, F. H., Proceedings of the National Academy of Science USA 77, 7380 (1980)), 2/78 (<3%) of pups born possessed tg DNA (ie had become transgenic). Following refinement, the efficiency of transgenesis by pronuclear microinjection in the mouse has now increased to a typical value of approximately 15%. However, the corresponding efficiency in other species, such as cattle, sheep, pigs and goats (examples of commercial species), is much lower (nearer 1%) This likely reflects the considerable facility with which mouse 1-cell embryos can be obtained and handled compared to difficulties with the 1-cell embryos of the other species. Unlike mouse 1-cell embryos, those of commercial breeds are opaque when viewed by standard light microscopy (due to their high lipid content). This is a significant draw-back to pronuclear microinjection because it requires that an injection needle be inserted precisely into a particular compartment of the egg (the pronucleus); the pronucleus must be located and therefore be visible. Attempts at circumventing this problem include the additional step of low speed centrifugation.

Transgenesis by pronuclear microinjection does not as yet permit the outcome of tg insertion to be controlled or predicted due to the quasi-random nature of integration site and number of copies integrated into the host genome. Greater control over the outcome of integration can be achieved by using (mouse) embryonic stern (ES) cell lines transfected with constructs capable of genome-targeted, homologous recombination Transfected ES cell lines can be selected and characterized in vitro to confirm the construct integration site. Reconstitution of embryos with such gene-targeted ES cells may then be used to produce chimaeric offspring. This method of genome modification is currently restricted to the one species for which established, germline-contributing ES cells exist—the mouse—with no demonstrated application to other species.

Limitations in the available strategies for modifying mammalian germlines have fueled a search for alternative methods. These include the use of recombinant retroviruses to infect oocytes or preimplantation embryos, replication-deficient adenovirus-mediated delivery systems and spermatozoa as vehicles for DNA delivery during in vitro fertilization. In this last approach live spernatozoa are used as a vector for the introduction of recombinant DNA into the oocyte in vitro; this system has triggered considerable controversy regarding its efficacy as a method of trazsgenesis. The biology of the phenomenon is poorly characterized and it is of limited use because of its unreliability.

It has previously been reported that following intracytoplasmic sperm injection (ICSI) into metaphase II mouse oocytes, sperm heads are able to support full development even though considered ‘dead’ in that they are immotile at the time of injection; of greater significance, membrane-disrupted spermatozoa are also able to support fill development (Perry, A. C. F., Wakayama, T. & Yanagimachi, R. Biology of Reproduction 60, 747 [1999]). Structures underlying the membranes of spermatozoa are highly conserved throughout biology. The major protein components of such structures predominantly the nucleus and perinuclear matrix) are positively charged, basic proteins. This suggests that they would support an electrostatic interaction with polyanions, such as nucleic acids, including DNA and RNA. These features have been utilized in the development of a novel method of transgenesis described herein This method offers marked advantages over existing methods: it yields transgenic offspring at a high efficiency; it may be used in the production of transgenic animals from a variety of different species; its flexibility permits the introduction of a broader spectrum of different types of tg and other molecules; it enables the manipulation of the biology of the unfertilized egg; it should support the subset of transgenesis events known as gene targeting, in which site-specific modifications are made within an entire genome. The method of the invention will hereinafter be described.

### SUMMARY OF THE INVENTION

The invention provides methods for introducing transgene (tg) nucleic acid (NA) into the cells of an organism, such as a plant or animal, or in vitro cultured cells, by injecting tg NA, or co-injecting tg NA that had previously been mixed with a nucleus, into an immature oocyte, an unfertilized oocyte or enucleated oocyte (known hereafter as “oocyte”).

In one embodiment of the invention, unfertilized oocytes are those arrested at the metaphase II (met II) of meiosis. Met II oocytes are at the stage that normally participate in fertilization in mammals. The invention further provides for the introduction of tg NA into a cell containing resident nuclear components, followed by activation of development.

As used herein, the term “transgene NA” (or “tg NA”) is intended to encompass any nucleic acid or its derivative that may be introduced into an oocyte to induce a change in the genomic sequence that was hitherto native to the host genome, thereby altering that genome. In one embodiment, the tg NA is deoxyribosenucleic acid, DNA The term “nucleus” used herein refers to the entire nucleus or that portion of the nucleus necessary for full embryonic development to term and beyond. In one preferred embodiment, the nucleus is the nucleus of a sperm call.

The invention further provides methods for producing a transgenic animal by co-inserting tg NA with a nucleus. In the method, the nucleus is exposed to the tg NA by, for example, mixing them together. In one embodiment, the nucleus is the nucleus of a spermatozoon whose membranes have been removed or disrupted. The invention allows for a variety of procedures of membrane disruption.

In a preferred embodiment of the invention, the nucleus is inserted into the oocyte by microinjection, and more preferably by piezo-electrically actuated microinjection. Use of a piezo-electrically actuated (as opposed to a conventional) pipette facilitates the microinjection process, rendering it swifter. This reduces cellular trauma, enhancing embryonic survival rate. The cell reconstituted in this way may be permitted to develop. In one embodiment, development produces a relatively homogeneous populations of cells (for example stem cells). In a further embodiment, the reconstituted cell is allowed to develop into a blastocyst following culture in vitro, and the resulting embryo may be transferred to a suitable surrogate mother at this or a previous stage in embryonic development, to permit full development.

We demonstrate herein the production of live transgenic offspring generated by the co-insertion of a sperm head with tg NA. We demonstrate not only the presence of the tg in offspring, but that it is expressed to alter the characteristics of the offspring. In different preferred embodiments of the invention, we show that sperm heads whose membranes have been disrupted promote transgenesis with a high efficiency. Membrane disruption may be achieved by different methods, including sperm treatment with detergent or by freezing or freeze drying. Sperm head membrane damage serves to indicate a sperm cell that is suitable for use in the method. We here use sperm heads, including membrane-damaged sperm heads, to demonstrate the principle of the invention. In one embodiment of the invention, damage to membranes allows tg DNA to gain access to sub-nuclear elements, including but not limited to the perinuclear matrix (in the case of spermatozoa), the nuclear matrix, chromatin and genomic DNA.

In one embodiment, the tg NA is a linear DNA fragment that encodes a readily detectable phenotypic marker. Transgenic embryos and offspring resulting from co-insertion of a nucleus with such DNA possess a genomic alteration that may alter their characteristics (phenotype) in a manner that is readily detectable. Examples of readily detectable markers that are suitable include firefly luciferase (Luc), Escherichia coli β-galactosidase (LacZ) and the Aequoria victoria green fluorescent protein (GFP).

Preferably, tg NA is mixed with the nucleus prior to co-injection using a micropipette. In another preferred embodiment, co-injection is into an oocyte arrested at metaphase II (met II) of meiosis. We here use DNA encoding either Aequoria victoria green fluorescent protein (GFP) or Escherichia coli β-galactosidase (LacZ) to demonstrate the principle of the invention, by showing that the tgs are expressed at a high frequency in transgenic embryos generated by the method of the invention In another embodiment, tg NA corresponds to an artificial chromosome such as a mammalian, yeast or bacterial artificial chromosome (MAC, YAC and BAC respectively Schindelhauer, D. Bioessays 21, 76 [1999]; Peterson, K. R. Methods in Enzymology 306, 186 [1999]; Kim, U. J., Bimen, B. W., Slepak, T., Mancino, V., Boysen, C., Kang, H. L., Simon, M. I. & Shizuya, H. Genomics 34, 213 [1996]). In a farther embodiment of the invention, tg NA corresponds to ribosenucleic acid (RNA) such as messenger RNA (mRNA), or to RNA-DNA heteroduplexes (chimaeras that possess at least one mismatch) or to peptide nucleic acids.

Thus, the invention provides an efficient method for producing transgenic offspring by the co-injection of a nucleus with tg DNA into an unfertilized oocyte. The invention is applicable to all organisms and collections of differentiated cells and stem cells, which can, or might be, generated following the insertion of a nucleus into an unfertilized oocyte. This includes the nuclei of cells derived from, without limitation, amphibians, fish, birds (eg domestic chickens, turkeys, geese and the like) and mammals, such as primates, ovines, bovines, porcines, ursines, caprines, felines, canines, equines, murines and the like. In one embodiment of the invention, the nuclei are from spermatozoa. The germane properties of sperm nuclei are conserved (see Kimura, Y., Yanagimachi, R., Kuretake, S., Bortkiewicz, H., Perry, A. C. F. & Yanagimachi, H. Biology of Reproduction 58, 1407 [1998]).

The co-introduction of tg DNA/nuclear material by microinjection is spatiotemporally distinct from methods that require cell fusion promoted either artificially or via in vitro (Lavitrano, M., Camaioni, A., Fazio, V. M., Dolci, S., Farace, M. G. & Spadafora, C. Cell 57, 717 [1989]). In one embodiment, the microinjection method entails first the selection of a nucleus (and NA) and subsequently its deposition into an oocyte by puncturing the plasma membrane of the oocyte.

Co-injection of tg DNA and nucleus by the method of the invention does not require that the nucleus be obtained from a living cell. This further distinguishes the method of the invention from claims exemplified by those wherein live sperm were mixed with tg DNA (in vivo or in vitro) and used to introduce the DNA via fertilization. Furthermore, co-injection of tg DNA and a nucleus from a membrane-disrupted cell according to the method of the invention, allows for the precisely controlled co-introduction of reagents that might be efficacious to the outcome of the procedure. Such a reagent may include an enzyme, antibody, or pharmacological signal transduction inhibitor that modulates recombination and/or embryo development to promote transgenesis. The introduction of the reagent into the oocyte may take place prior to, during, or after the co-introduction of tg NA and nucleus.

### BRIEF DESCRIPTION OF DRAWINGS

The invention will hereinafter be described in detail with reference to the accompanying drawings in which:

FIG. 1 shows representative sagittal sections through the heads of mouse spermatozoa that were either intact (‘fresh’)(A), or whose membranes had been disrupted by triton X-100 (B), freeze-thawing (C) or freeze-drying (D) as per Example 1. Abbreviations are: ac, acrosomal cap; eq, equatorial segment; pa, postacrosomal region. Plasma and acrosomal membranes (except for those in the equatorial region) are absent or disrupted. Disruption is clearest in the membranes of the acrosomal cap.

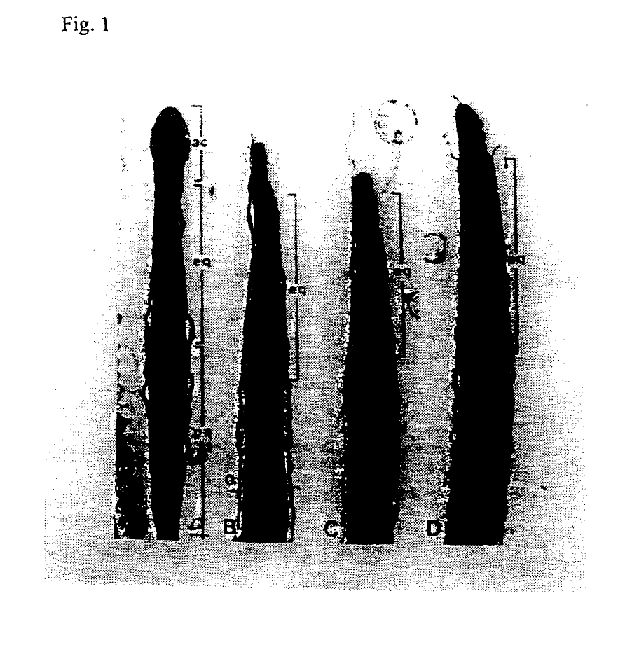

FIG. 2 shows transgenic embryos produced by single-shot double transgenesis. Oocytes were microinjected with spermatozoa that had been preincubated with a mixture of pCX-LacZ and pCX-EGFP tg DNAs as per Example 3. Panels show the same embryos (×400) after 3.5 days viewed by Hoffman modulation contrast microscopy (A) unstained, (B) for GFP expression under long-wavelength (480 nm) UV light, and (C) stained with X-Gal for β-galactosidase expression.

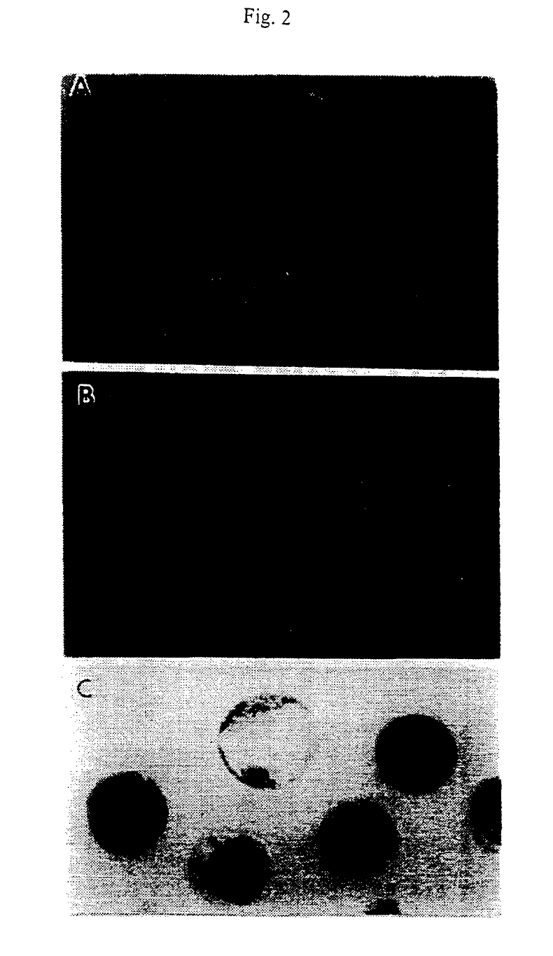

FIG. 3 shows an analysis of tail-tip biopsies from transgenic founders and non-transgenic controls. (A) Fluorescent stereomicroscopy (×40) of tail tips from non-transgenic (a; mouse #16) and transgenic green-fluorescent (b; mouse #3) lines. Green fluorescent skin could be visualized through non-green hairs. (B) Southern blot analysis (showing estimated tg copy numbers per genome in parentheses) of total DNA from control B6D2F₁ (0), #3 (5-9), #19 (>50), #28 (5-9) and #41 (2) using a pCX-EGFP fragment as probe. (C) Polymerase chain reaction (PCR) analysis of total DNA from #16, #17, #30, #36, #47, #49, control B6D2F₁, #3, #19, #28, #41. See Example 5.

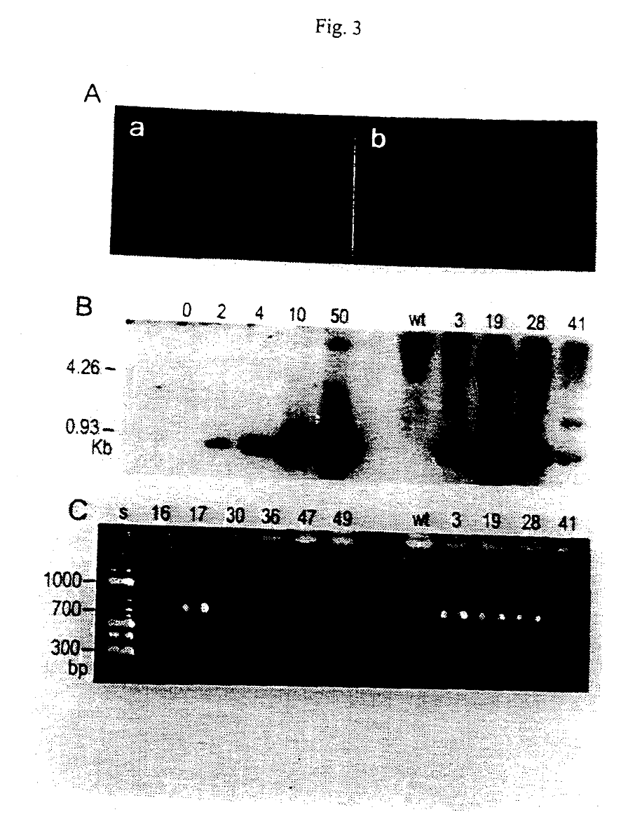

### DETAILED DESCRIPTION OF THE INVENTION

### Method of Performing Transgenesis

The present invention describes a distinctive set of methods for generating cells harboring an integrated transgene. The method of the invention comprises the steps of: (I) Exposure of exogenous, tg NA to nuclear components (a nucleus, or portion thereof, including the chromosomes); (II) Microinjecting the tg NA-nucleus mixture or tg NA into an unfertilized egg; (III) Allowing the resulting cell to develop. Development may be to produce differentiated cells or stem cells to an embryo, or to an individual following embryo transfer into a surrogate mother. We now present the individual steps in greater detail and show how they are distinctively arranged in respect of one to the other in the present invention.

### I. Exposure of Exogenous, tg NA to Nuclear Components

The method allows for exposure of exogenous, tg NA to nuclear components, prior to microinjection. Nuclei may be from somatic or other cells. Exogenous, tg NA may be introduced into cells by methods exemplified by, but not limited to, electroporation, lipofection, transfection, mixing or microinjection prior to the time of nucleus insertion. These methods are well known to those skilled in the art.

In one embodiment of the invention, a fragment of pCX-EGFP DNA (see Examples 1 & 2) was mixed for 30-180 seconds on ice or 25° C. by triturating with non-transgenic sperm whose membranes had been disrupted by treatment with a detergent, such as, but not limited to triton X-100 (3-[3-cholamidopropyl)dimethylammonio] 1-propanesulfonate) (CHAPS), sodium dodecylsulfate (SDS), sodium lauryl sulfate (SLS), mixed alkyltrimethylammonium bromide (ATAB), and the like. In further embodiments of the invention, sperm membrane disruption was by freezing/thawing or by freeze-drying. These methods cause sufficient damage to sperm to decapitate a proportion of the population. The extent of membrane damage increases in the order: freshly isolated sperm<triton X-100<freeze/thaw<freeze-dry.

One manner in which the method of this invention allows significant advantages over previous methods is by extending the choice of tg NA. Tg NA may include single-stranded or double-stranded RNA or DNA, chimeric heteroduplexes (such as RNA-DNA hybrids; see below) and relatively large molecular species such as chromosomes. In one embodiment of the invention, the NA may correspond to DNA molecules which are large (for example 50 kilobase pairs [kb] to >1 megabase pairs). Examples of large DNA molecules include, but are not limited to, artificial chromosomes such as mammalian, yeast and bacterial artificial chromosomes (MACs, YACs and BACs respectively). Such large molecules are sensitive (relative to small molecules) to damage, especially during micromanipulation. Hence, the step of gentle mixing with a membrane-challenged sperm head provided in one embodiment of the invention likely allows stabilization of large DNA molecules by (i) allowing them to associate with the relatively massive protective structure of the sperm head, and (ii) not subjecting them to chemical or physical forces (such as shear forces) during the injection procedure, thereby increasing the success rate.

A further manner in which the method of the invention permits significant advantages over previous methods is that it provides means by which novel properties of the cytoplasm of the unfertilized (met II) oocyte may be harnessed. In particular, nuclear decondensation following microinjection into met II oocytes provides a situation in which genomic DNA is relatively exposed and therefore more reactive (described in Perry, A. C. F., Wakayama, T. & Yanagimachi, R. Biology of Reproduction 60, 747 (1999). Moreover, the met II oocyte contains recombinogenic factors, since the injection of mouse met II oocytes with damaged and spermatozoa heated to 48C results in recombination to produce bicentric (ic translocated), acentric, and ring chromosomes, and chromosome fragments (Ward, W. S., Perry, A. C. F., & Balhorn, R. Biology of Reproduction, accepted for publication). The method of the invention thus provides for gene targeting via homologous recombination and/or by recruiting factors responsible for DNA repair, as, for example in chimaeroplasty (see below). In one embodiment, the invention allows for such recombination to be enhanced by the inclusion of site-specific and non-site-specific recombination-promoting agents in the nucleus-NA mixture. Such agents include, without limitation, Escherichia coli RecA protein, the human RecA counterpart, HsDmc-1, single-stranded DNA binding proteins such as bacteriophage T4 gene 32 product, site specific recombinase(s) (such as Cre and Flp recombinases) and the like. In a further embodiment, the NA used is a gene targeting DNA construct containing extensive sequences that match those of a small number (usually 1) of genomic loci native to the genome. The design criteria for gene targeting vectors (for example, as used to ‘knock out’ genes in embryonic stem cells) are well established and known by those skilled in the art.

The method of the invention described here further allows that the NA to correspond to a chimaeraplast. In one embodiment, these molecules are short (<100 nucleotides) RNA-DNA heteroduplexes (for example, Yoon, K., Cole-Strauss, A. & Kmiec, E. B. Proceedings of the National Academy of Sciences USA 93, 2071 [1996]) which contain the near-perfect compliment of a genomic sequence with the exception of a 1-3 base mismatch near the center. Such molecules can direct cellular DNA repair machinery to introduce the mismatch into the genomic sequence. In this embodiment of the invention, the DNA repair machinery is within the oocyte, such that site-specific mutations may be introduced during, or following, nucleus-chimaeraplast co-injection into the met II oocyte, or injection of a chimaeraplast in the absence of a nucleus.

The invention allows for the inclusion of additional substances during mixing. These may include, but are not limited to, modulators of nuclease activity (eg ethylenediamine tetraacetic acid [EDTA], aurintricarboxylic acid, restriction endonucleases and the like), apoptosis (eg aurintricarboxylic acid, and the like), proteolysis (eg leupeptin, E.64 and the like), and DNA binding proteins such as protamines and topoisomerases. In one embodiment of the invention, supplementary agents are mixed with tg NA in the absence of a nucleus. In this case, the genome is provided by a nucleus already resident within the oocyte microinjected with the tg NA, as for example in the case of an enucleated met II oocyte that had (subsequent to enucleation) received the nucleus of a somatic cell via nuclear transfer, or a non-enucleated met II oocyte. These cells are activated to undergo development artificially using parthenogenetic agents (in the presence of cytokinesis blocking agents such as cytochalasin B, cytochalasin D and the like) by methods known to those skilled in the art.

Nucleus-NA mixtures are held for a period of time to allow nucleus-NA association. In one embodiment, the association is given at least 30 sec to occur. The mixture is then transferred to a microscope stage for microinjection. In a further embodiment, microinjection is completed within 1 h of nucleus exposure to NA.

### II. Microinjecting the tg NA-Nucleus Mixture or tg NA into an Unfertilized Egg

Nuclei which have been exposed to, tg NA are inserted into an unfertilized oocyte by microinjection. Nucleus-NA mixtures are transferred to a droplet on the microscope stage of the microinjection unit such that they may be gathered into the microinjection needle for injection. In one embodiment, nucleus-NA mixtures are supplemented with a solution of polyvinylpyrrolidone to aid manipulation. Collection of the tg NA-nucleus sample to be injected is by aspiration into the injection pipette. In a preferred embodiment of the invention, the microinjection needle is piezo-actuated. A suitable piezo electric driving unit is sold by Prime Tech Ltd. (Tsukuba, Ibaraki-ke, Japan) and Eppendorf Scientific (New York, USA) and employed according to the instructions of the vendor. The unit is capable of transmitting a piezo-electric pulse to advance the microinjection pipette tip to which it is attached a very short distance (of the order of 0.5 μm) in a highly controlled and rapid manner. The intensity and interval between each pulse (which may be varied on the control unit, with typical values of 1-5 for intensity and 1-16 for so) are applied to advance the tip through the zona pellucida of the oocyte (whilst fixing the oocyte in place using light suction from a holding pipette). The pipette tip is the apposed to the oocyte plasma membrane and advanced (toward the opposite face of the oocyte) until the oocyte plasma membrane is deeply invaginated Upon application of a small number (typically one) piezo pulses (typically, intensity 1-4, speed, 1) the plasma membrane is punctured, allowing expulsion of tg NA-nucleus or tg NA into the cytoplasm of the oocyte. In one embodiment, injection is through a flush-ended borosilicate glass needle (of typical internal diameter, 4.5-10 μm) that contains mercury near its end; the mercury increases the momentum and control of the piezo-actuated needle tip. Alternative microinjection variants may be used to insert the tg NA and/or nucleus, including conventional pipettes as exemplified in the description of Yanagida, K., Yanagimachi, R., Perreault, S. D. and Kleinfeld, R. G. Biology of Reproduction 44, 44 (1991).

Injection of nuclei may be into an oocyte from the same species, or from an oocyte taken from a different species. Furthermore, the method of the invention allows for NA injection, or NA-nucleus coinjection, into oocytes, enucleated oocytes, or immature (eg germinal vesicle stage) oocytes, including pre-ovulatory oocytes that have been matured in vitro. In vitro maturation of oocytes (IVM) is desirable where sources of mature oocytes are limited or non-existent and may be in the presence of agents which render them more suitable for microinjection. Bovine oocyte IVM has been described in WO 98/07841 and for mouse oocytes in Eppig & Telfer (Methods in Enzymology 225, 77, Academic Press [1993]). Mature oocytes may be obtained by inducing super-ovulation following the sequential administration of gonadotrophic or other hormones (for example, the sequential administration of human chorionic gonadotrophin and pregnant mare serum gonadotrophin) and the subsequent surgical harvesting of ova (eg 80-84 hours after the onset of estrous in the cat, 72-96 hours in the cow and 13-15 hours in the mouse).

In an additional embodiment of the invention, the NA-exposed nucleus is from a diploid somatic cell, and is inserted into the cytoplasm of an oocyte whose chromosomes had been removed (an enucleated oocytc); the method of oocyte enucleation is well known to those skilled in the art and is utilized, for example, in Wakayama, T., Perry, A. C. F., Johnson, K., Zuccotti, M. & Yanagimachi. R. Nature 394, 369 (1998). Collection of nuclei is from cells that had been dispersed, for example by treatment with with a mixture of trypsin (0.025%) and ethylenediamine tetraacetic acid (EDTA; 0.75 mM). Cells are artificially stimulated to initiate development 0-6 h after reconstitution, using an stimulus such as, but not limited to. Sr²⁺, ethanol or an electric pulse, according to known methods The method of the invention applies to nuclei taken from the cells of amphibians, fish, birds (eg domestic chickens, turkeys, geese and the like) and mammals, such as primates, ovines, bovines, porcines, ursines, caprines, felines, canines, equines, murines and the like, either grown in vivo or in vitro.

In a further embodiment of the invention, the nucleus is contained within a sperm head such as a membrane-challenged sperm head. Injection of a sperm head efficiently activates the injected oocyte sufficient for full development to term, following transfer of the developing embryo to a surrogate mother by a procedure known to those skilled in the art. The sperm head may be treated by heat or another agent that ablates its ability to activate an oocyte, in which case oocytes are subjected to an activating stimulus following microinjection, to induce development Spermatozoa may be from amphibians, fish, birds (eg domestic chickens, turkeys, geese and the like) or mammals, such as primates, ovines, bovines, porcines, ursines, caprines, felines, canines, equines, murines and the like.

The method of the invention allows for a large range of injection pipette tip diameters. Previous methods of transgenesis either do not permit the introduction of large segments of DNA (for example, where viral vectors are employed) or else may permit their introduction but with considerable technical difficulties. For example, the method of pronuclear microinjection is not suited to the high viscosity of preparations of artificial chromosomes; injection through the fine pipettes used (1-2 μm tip diameter) is difficult and DNA molecules often shear, resulting in failure. Furthermore, handling is difficult, with such narrow tips tending to become more sticky after fewer uses. Contrastingly, pipettes used in the method of the invention now described are typically of tip diameter >5 μm. The relatively large tip diameter (i) renders the handling of viscous DNA solutions easier, partly because needles are less sticky, and (ii), generates lower magnitude sheer forces; large DNA molecules are sensitive to damage by sheer.

A further advantage of the method of the invention is that it does not demand the injection of materials into a precise location within the oocyte. This is in contrast to pronuclear microinjection. This advantage is especially relevant to species whose oocyte cytoplasm is lipid-rich and thus opaque to light microscopy, such as the oocytes of many commercial species and breeds. Hence, the method of the invention does not require that the spatial relationship between the microinjection pipette tip and the oocyte cytoplasm be known precisely during microinjection.

The method of the invention allows the injection of tg NA in the absence of a co-injected nucleus. In one embodiment, tg NA is first mixed with components which stabilize the NA such as, but not limited to, basic proteins derived from sperm (eg protamines, perinuclear components and the like). In this case, injection is into a cell which possesses a nucleus. Such cells are exemplified by a met II oocyte (in which case the resulting embryo is a parthenogenote) or an enucleated met II oocyte into which a somatic or other nucleus had been implanted via nuclear transfer as described in Wakayma, T., Perry, A. C. F., Johnson, K., Zuccotti, M. & Yanagimachi, R. Nature 394, 369 (1998), in which report the embryos are clonally derived. Oocytes are artificially stimulated to initiate development using a stimulus such as, but not limited to, Sr²⁺, ethanol or an electric pulse, according to known methods.

### III. Allowing the Resulting Cell to Develop

Microinjected cells are allowed to develop either following removal to suitable culture conditions in vitro, or to a suitable surrogate mother. In some cases it is desirable to culture the cells to develop into an embryo, and in a further embodiment of the invention, to examine embryos that had been cultured in vitro such that their development can be described and tg expression determined. Hence, the method of the invention permits the selective transfer of transgenic embryos if they contain a tg whose expression can be monitored without killing the embryo. This is the case for GFP whose expression is driven in early embryos, as is the case for CMV-IE enhancer/chicken b-actin promoter combination. Expression may be monitored simply by viewing embryos briefly under long-wavelength (480 nm) ultra-violet UV illumination. Exposure to long-wavelength UV light is minimized to reduce potential UV damage to embryos, which damage may impair subsequent development of the embryo.

Methods of embryo culture and transfer are well known to those skilled in the art. Following either selective or non-selective (that is, not necessarily based on tg expression) embryo transfer to surrogate mothers, pregnancies are supported to term, resulting in the birth of live transgenic offspring. Confirmation of tg integration in offspring is by physical analysis of genomic DNA using methods that include Southern blotting and polymerase chain reaction and which are well known to those skilled in the art. Transgenic expression in offspring may be discernible as a character trait (ie phenotypically) or by analysis of tissues immunologically or by mRNA analytical methods such as Northern blotting. Ectopic expression of GFP in transgenic pups is readily discerned by examining the skin of new-born offspring illuminated by long-wavelength UV.

### EXAMPLES

The following examples are intended to illustrate the method of the invention without seeking to limit the scope of the invention.

### Reagents

All compounds were purchased from Sigma chemical Co (St Louis, Mo.) unless stated otherwise.

### Animals

Oocyte donors (B6D2F1), sperm donors (B6D2F1) and foster mothers (ICR) were maintained within the guidelines of the Laboratory Animal Service at the University of Hawaii and those prepared by the Committee on Care and Use of Laboratory Animals of the Institute of Laboratory Resources National Research Council (DHEW publication no. [NIH] 80-23, revised in 1985). Animal handling protocols were as reviewed and approved by the Animal Care and Use Committee at the University of Hawaii.

### Preparation of Oocytes

Mature oocytes were collected from the oviducts of pregnant mare serum gonadotrophin-primed (5 IU), superovulated, 4-10-wk-old female B6D2F1 mice 14.5-16 h after the intraperitoneal administration of 5 IU human chorionic gonadotrophin (hCG). The cumulus cell mass was dispersed by immediate treatment in CZB-H (CZB buffered with 20 mM HEPES, pH7.4; Chatot, C. L., Lewis, J. L., Torres I. & Ziomek. C. A. Biology of Reproduction 42, 432 [1990]) containing 0.1% (w/v) bovine testicular hyaluronidase (300 U/mg, ICN Biochemicals, Costa Mesa, Calif.) for 5-10 min at room temperature. Cumulus-free oocytes were washed four times in CZB-H and transferred to a drop of CZB under mineral oil (Squibb & Sons, Princeton, N.J.) equilibrated in 5% (v/v in air) CO₂ at 37° C.

### Manipulation of Mouse Eggs and Embryos

Piezo-electrically actuated microinjection of sperm heads into mouse eggs has been described by Kimura Y. & Yanagimachi, R. Biology of Reproduction 52, 709 (1995). Injections were usually completed within 18-19 h post hCG administration. The injection needle tip diameter was typically 5 μm. Test solution (usually with a single sperm head) was drawn into the pipette following expulsion of a small amount of mercury into the test solution droplet. This ensured that ahead of the mercury boundary, the test solution filled the pipette and was therefore not diluted ether. The equivalent of approximately 1 pl was delivered into the ooplasm per microinjection. Injected oocytes were maintained in operation medium (CZB-H) for approximately 2-10 min prior to transfer to CZB under mineral oil equilibrated in 5% (v/v in air) CO₂ at 37C. Where appropriate, oocytes were artificially activated by incubation, immediately after injection, for 45-60 min in Ca²⁺-free CZB containing 6.7 mM SrCl₂, under mineral oil equilibrated in 5% (v/v in air) CO₂ at 37° C. After this time, eggs were washed briefly in, and transferred to, fresh CZB, and incubation continued. Culture in vitro of embryos was in CZB for up to 4 days. Two-cell embryos (following approximately I day of culture) or morulae/blastocysts (following 2.5 -3.5 days of culture) were transferred to the oviducts of pseudopregnant albino ICR/CD1 mice that had been mated with vasectomized males of the same strain on the evening of microinjection. Births resulting from in vitro manipulation were thus characterized by their black eyes and coat color.

### Example 1

### Preparation and Microinjection of Sperm Nuclei

The isolation and culture of B6D2F₁ mouse metaphase II oocytes for microinjection was essentially as described previously. Spermatozoa were collected from mature 136D2F₁ male mice in 400 μl CZB medium. Isolation of spermatozoa, for triton X-100 extraction was by finely chopping two caudae epididymides at 0-1° C. in Nuclear Isolation Medium (NIM: 125 mM KCl; 2.6 mM NaCl, 7.8 mM Na₂HPO₄, 1.4 mM KH₂PO₄, 3.0 mM EDTA; pH7.45) and filtering the resulting sperm suspension to produce a final volume of 900 μl. Piezo-actuated microinjection of oocytes and culture of embryos in CZB under mineral oil equilibrated in 5% (v/v in air) CO₂ at 37° C. has been detailed elsewhere. For microinjection, sperm heads were aspirated into a pipette attached to a piezo electric pipette-driving unit and one injected per oocyte. Oocytes that lysed soon after injection were discarded. Where appropriate, dislocation of heads from tails was by the application of a single piezo pulse to the region of the sperm mid-piece. This procedure of itself disrupts membranes and thus represents a difference between the ‘fresh’ spermatozoa used here and previous reports of live spernatozoa promoting transgenesis via in vitro fertilization. We estimate that approximately 1 pl was displaced from the pipette interior per injection.

### Example 2

### Exposure of Sperm Nuclei to GFP or β-galactosidase tg NA by Mixing: Production of Transgenic Embryos

The large (3.5 kb) SalGI-BamHI figment of plasmid PCX-EGFP used here harbors a GFP gene expressed from a strong CMV-IE/chicken β-actin enhancer-promoter combination (Niwa, H., Yamamura, K. & Miyazaki, J. Gene 108, 193 [1991]), but lacks a eukaryotic origin of replication (Zhang, G. Vanessa, G. & Kain, S. R. Biochemical and Biophysical Research Communications 227, 707 [1996]; Takada, T. Iida, K. Awaji, T. Itoh, K. Takahashi, R. Shibui, A. Yoshida, K. Sugano, S. & Tsujimoto, G. Nature Biotechnology 15, 459 [1997]). Sperm nuclei were either mixed with pCX-EGFP fragment without further preparation (‘fresh’), or after they had been subjected to one of three membrane-disruption protocols: freeze-thawing (Wakayama, T., Whittingham, D. G. & Yanagimachi, R. Journal of Fertility and Reproduction 112, 11 [1998]), freeze-drying (Wakayama, T. & Yanagimachi, R. Nature Biotechnology 16, 639 [1998]) or triton X-100 extraction. For triton X-100 extraction, 100 μl 0.5% (v/v in NIM) triton X-100 was added to 900 μl sperm suspension in NIM (see example 1) and mixed by trituration for 30 sec on ice. Cells were pelleted by centrifugation for 1 min at 20,000 g, 2° C. and thoroughly resuspended in 2 ml ice-cold NIM before repelleting for 2 min at 20,000 g, 2° C. This final pellet was resuspended in 400 μl CZB or NIM. Prior to microinjection, 1 μl of the DNA fragment was mixed with 9 μl sperm suspension (containing 2-5×10⁵ spermatozoa in CZB or NIM) by pipetting to give a final DNA fragment concentration of 7 ng/μl. The DNA/sperm mixture was incubated at room temperature (approximately 25° C.) or on ice for 1 min. It was then mixed with a polyvinylpyrrolidone (PVP; average Mᵣ 360,000) solution to give a final concentration of approximately 10% (wlv) PVP and placed on the microscope stage for microinjection. All injections were performed in CZB-H at room temperature within 1 h of sperm-DNA mixing, or within 1 h of sperm-triton X-100 mixing.

Embryos were examined 3-3.5 days after microinjection by epifluorescence microscopy for expression of GFP using a UV light source (480 nm) with FITC filters. This enabled the clear identification of non-fluorescent (ie non-GFP-expressing), weakly-fluorescent and strongly-fluorescent embryos and mosaic embryos (containing both fluorescent and non-fluorescent cells), which were scored accordingly.

Analogous experiments exemplifying the method of the invention used a different tg NA. Purified lacZ-harboring fragments of pxCANLacZ, linearized by digestion either with SalGI or XhoI and SalGI, were mixed with spermatozoa at concentrations of 4.5 and 9 ng/μl respectively and microinjected as described above. Both lacZ fragments gave similar results; the pxCANLacZ XhoI-SalGI fragment lacks a eukaryotic replication origin. The β-galactosidase encoded by pxCANLacZ contains a nuclear localization signal. Assessment of pxCANLacZ β-galactosidase expression in day 3 embryos was performed after a 5 min fixation at room temperature in phosphate buffered saline, pH7.6 (PBS), containing 1% (v/v) formaldehyde, 0.2% (v/v) glutaraldehyde, 5 mg/ml bovine serum albumin (BSA). Fixed embryos were washed thoroughly in PBS containing BSA (5 mg/ml) and stained by incubation for 5 hours at 37° C. in PBS containing 5 mg/ml BSA, 4 mM potassium ferricyanide, 4 mM potassium ferrocyanide, 2 mM MgCl₂ and 1 mg/ml 5-bromo-4-chloro-3indolyl-β-D-galactopyranoside (X-gal). Embryos were examined and scored by light microscopy.

Results, presented in Table 1 below, demonstrate that the method of the invention generates transgenic embryos with a high efficiency. The method yielded detectably tg expressing embryos when the pCX-EGFP DNA fragment concentration was 500 pg/μl but not 50 pg/μl. This suggests that the method is sensitive at average DNA concentrations corresponding to as few as approximately 15-150 molecules per injection.

TABLE 1
In vitro culture and tg expression of embryos
produced after microinjection of metaphase II oocytes
with exogenous reporter-encoding DNA and sperm heads.

| | | | Total morulae-blastocysts (m-b) | | | |
|---|---|---|---|---|---|---|
| | | | and fluorescence (GFP) | | | |
| | Sperm | No. | or staining (LacZ) on day 3 | | | |
| Fragment† | treatment‡ | oocytes | m-b (%)* | −§ | +/−§ | +*§ |
| pCX-EGFP | None (fresh) | 162 | 134 (83)^{a} | 100 | 13 | 21^{a} |
| pCX-EGFP | Triton X-100 | 270 | 212 (79)^{a} | 75 | 37 | 100^{b} |
| pCX-EGFP | Freeze-thaw | 313 | 155 (50)^{b} | 28 | 31 | 96^{b} |
| pCX-EGFP | Freeze-dry | 278 | 154 (55)^{b} | 20 | 23 | 111^{b} |
| px-CANLacZ | Freeze-thaw | 151 | 110 (73)^{a} | 7 | 45 | 58^{b} |
| px-CANLacZ | Freeze-dry | 136 | 108 (78)^{a} | 8 | 32 | 66^{b} |
| pCX-EGFP | Washed | 153 | 114 (75)^{c} | 43 | 4 | 67^{c} |
| pCX-EGFP | Not washed | 117 | 83 (71)^{c} | 17 | 3 | 63^{c} |
| | \mathring{1} | | | | | |
| | Freeze-thaw | | | | | |
| → \mathring{2} | | 71 | 56 (79) | 56 | 0 | 0 |
| pCX-EGFP | | | | | | |
| \mathring{1} | | | | | | |
| pCX-EGFP → | | | | | | |
| | \mathring{2} | 51 | 35 (69) | 35 | 0 | 0 |
| | Freeze-thaw | | | | | |
| pCX-EGFP | — | 49 | 48 (98) | 48 | 0 | 0 |
| alone | | | | | | |
| *When values with superscripts a and b within the same column are compared, they differ significantly (P < 0.05). Values c within the same column do not differ significantly. | | | | | | |
| ^{†}Exogenous DNA fragments were pCX-EGFP-BamHI-SalGI or pxCANLacZ-SalGI, SalGI-Xhol, or XhoI. Fragments were mixed with sperm heads at DNA concentrations of 5-10 ng/l. With the exception of the last three rows (see Example X), exogenous DNA was injected after mixing with sperm samples as described in Example 2. | | | | | | |
| ^{‡}Sperm treatments are as described in example 1. Preparation of washed and non-washed samples is described in Example 4. As a negative control, all experiments included same-day injection of a fresh aliquot of the appropriate sperm preparation mixed with NIM or CZB alone; following culture, no ‘false positive’ expression was ever observed. Serial injections were separated by 30-90 min per pair, with heads prepared by the freeze-thaw method (same-day positive control co-injection | | | | | | |
| # of freeze-thaw sperm pre-mixed with DNA yielded fluorescent blastomeres in 75% of embryos) as described in Example X. Injection of tg DNA alone was followed by parthenogenetic activation as per Example X. | | | | | | |

§Tg expression: −, negative; +, positive; +/−, m-b containing both + and − cells (mosaics).

### Example 3

### Production of Double GFP and β-Galactosidase tg Embryos in One Manipulation (Single Shot Double Transgenesis)

Single-shot double transgenesis was used to generate embryos co-expressing two tgs after a single microinjection as described in Example 1, with the following modifications. Sperm heads were co-injected with a DNA solution containing: 2.5 ng/μl pCX-EGFP SalGI-BamHI fragment and 2.5 ng/μl pCX-LacZ SalGI-PstI fragment. pCX-LacZ is a derivative of pCX-EGFP in which the EGFP gene is replaced by one encoding β-galactosidase. Following culture in vitro, embryos were first scored for GFP expression and then for β-galactosidase expression as described in examples 1 and 2 respectively. For photography, embryos were mounted between a microscope slide and cover slip and images collected to show development and GFP expression, prior to fixation and staining to show LacZ expression.

### Example 4

### Production of GFP tg Embryos Following Washing of the tg NA-Nucleus Mixture

The sperm suspension in each washing experiment was divided into two 5 μl aliquots immediately after mixing and incubating with pCX-EGFP DNA for 1 min. One aliquot (washed sperm) was diluted and washed by mixing well with 50 μl ice-cold, fresh CZB or NIM. Both aliquots were then pelleted for 2 min at 20,000 g, 2° C. The supernatant from the washed sperm aliquot was carefully removed and replaced with 5 μl fresh CZB or NIM; the supernatant from the second aliquot was used to resuspend its own pellet (this sample was therefore not washed). Results, presented in Table 1, are strongly suggestive of the ability of a nucleus and NA to associate in vitro prior to microinjection.

### Example 5

### Production and Confirmation of GFP tg Pups and Confirmation of Their Transgenic Nature

To determine whether genomic integration of tg DNA constructs could be demonstrated in live offspring, sperm heads that had been subjected to one of the three membrane disruption procedures were co-injected with pCX-EGFP DNA, the resulting embryos were cultured in vitro for up to approximately 3.5 days (to the morula-blastocyst stage) and embryos were then transferred to surrogate mothers non-selectively (ie not on the basis of fluorescence). Phenotypic analysis of tg integration was by examination of offspring under long-wave UV. A high proportion (17-21%) of offspring were transgenic with respect to observable GFP expression in skin (Table 2 Below); this efficiency did not depend on the membrane disruption method used to prepare spermatozoa. Rates of zygotic development to term were comparable for each of the three groups of membrane-disrupted sperm heads (12-14%).

TABLE 2
Development of phenotypically transgenic (green) pups and their siblings.

| Sperm | | | Total | |
|---|---|---|---|---|
| treatment* | No. oocytes | m-b transferred¶ | pups | ⁺(green)pups§ |
| Freeze-dry | 116 | 67(4) | 14 | 3^{a} |
| Freeze-thaw | 97 | 53(3) | 12 | 2^{a} |
| Triton X-100 | 218 | 150(9) | 31 | 6^{a} |

*Each row records development of embryos and pups produced from oocytes co-injected with demembranated sperm heads and a fragment of plasmid pCX-EGFP (see example 5).
^{a}Values do not significantly differ.
¶m-b, morulae-blastocysts. Values in parentheses show the number of surrogate mothers used as recipients in embryo transfers
§Tg expression: ⁺, positive pups are those expressing GFP ectopically in thor skin.

In a further experiment, a single β-galactosidase-expressing pup was born following microinjection of a demembranated sperm head had been exposed to pCX-LacZ (see Example 3) according to the method of Example 2. β-galactosidase (LacZ) expression was demonstrated following fixation and X-Gal staining of a tail-tip biopsy (obtained 3 days post natum) according to the method of Example 2. This demonstrates that the method of the invention is not restricted to one tg type and suggests the applicability of the method to a variety of tgs.

Tail-tip biopsies from 3 to 6 week-old, randomly-selected green pups and their non-green litter-mates were used for extraction of total, genomic DNA. Photography of tails was under a fluorescent stereomicroscope equipped with a 480/40 nm filter. In Southern analysis, 10 μg genomic DNA per sample was digested with EcoRI and probed with the 733 bp EcoRI fragment of pCX-EGFP. Oligonucleotide primers used for the detection of the GFP gene by PCR of 1 μg genomic DNA per reaction were forward (TTGAATTCGCCACCATGGTGAGC) and reverse (TTGAATTCTTACTTGTACAGCTCGTCC). Reaction parameters were 95° C. for 9 min (1 cycle); 94° C. for 45 sec, 60° C. for 30 sec, 72° C. for 45 sec (40 cycles). Electrophoretically separated products were visualized following ethidium bromide staining.

Physical analysis of tail tip genomic DNA by Southern blotting or polymerase chain reaction showed that all founder lines (ie first generation transgenic animals) that exhibited green fluorescence possessed the tg, including one initially scored phenotypically negative but whose biopsied tail tip exhibited GFP expression. In three cases, the tg was demonstrated by PCR in founders that lacked detectable green fluorescence, with non-expression presumably being due to locally cis-active elements at the tg integration locus. Southern analysis indicated that tg copy numbers in founders ranged from ≦1->50; this result resembles the pattern of tg integration after pronuclear microinjection. Both the physical characterization of genomic pCX-EGFP DNA and the efficiency of GFP expression suggest that tg DNA did not undergo gross rearrangements on integration.

A random selection of 12 GFP-expressing founders (8 females, 4 males; from Table 2 and analogous series) were crossed with non-transgenic animals and produced litters in all but one case (female). Of the 11 fertile founders, 8 produced pups expressing GFP ectopically in their skin with a frequency of 27-50% (average=40%). The pattern of tg inheritance was in most cases consistent with Mendelian gerinline transmission of a single locus GFP gene.

### Example 6

### Use of the Cre-lax System to Monitor Transgenesis

It is desirable to score embryos generated by the method of the invention for tg integration without the necessity for persistent tg expression throughout the life of the transgenic individual. This may be achieved using tg vectors that contain an internal ribosome entry site (IRES). Embryos are generated by microinjection as described in Example 1. The DNA construct contains the GFP gene expressed from a strong CMV-IE/chicken β-actin enhancer-promoter combination of Example 2, flanked by lox sites. This enhancer-promoter is adjacent to a second element sufficient to drive the programmed expression of any given tg. The GFP open reading frame is adjacent to the given tg open reading frame, separated by an IRES. The tg DNA construct is mixed with demembranated spermatozoa and coinjected into the met II oocyte of a strain expressing the Cre recombinase under the control of, for example the goosecoid gene promoter, which functions at the gastrulation stage of development, thereby excising the CMV-IE/chicken β-actic enhancer-promoter element during that stage. Embryos are cultured in vitro for up to approximately 3.5 days (to the morula-blastocyst stage) and transferred to surrogate mothers selectively (ie on the basis of fluorescence) to enable their full development.

### Example 7

### Microinjection of tg DNA in the Absence of a Sperm Nucleus Suggests that Sperm Nuclear Components Sustain tg DNA in a Recombinogenic Form

To probe whether NA integration could occur inside the oocyte (ie after microinjection), we injected sperm heads and pCX-EGFP DNA serially, with no mixing prior to injection (Table 1). We consistently failed to observe exogenous tg (GFP) DNA expression, even though 75% positive control embryos (freeze-thaw sperm head-pCX-EGFP co-injection as for Table 1) were fluorescent.

A fresh dilution of the SalGI-BamHI fragment of plasmid pCX-EGFP (7 ng/μl in NIM) was mixed with an equal volume of PVP 20% (w/v) and approximately 1 pl injected per oocyte. Following a recovery time of 5-10 min at room temperature, oocytes were transferred to Ca²⁺-free CZB containing 10 mM SrCl₂ and the cytokinesis-blocking agent, cytochalasin B at 5 μg/ml, and incubated for 6 h at 37° C. (Bos-Mikich, A, Whittingham D. G. & Jones, K. T. Developmental Biology 182, 172 [1997]). They were then transferred to CZB and incubation continued under standard embryo culture conditions. Embryos were scored for GFP expression after 3.5 days as described in Example 2. In contrast to co-injection with a sperm head, injection of a similar quantity of GFP tg DNA alone did not preclude good parthenogenetic development (98% of oocytes surviving injection developed to the morula-blastocyst stage). Moreover, none of the resulting embryos exhibited observable tg expression (Table 1). Hence, in the absence of sperm heads there could have been little tg expression or epichromosomal persistence of transcriptionally active tg DNA.

We observed mosaic embryos containing both GFP-positive and -negative blastomeres (±morulae-blastocysts) after sperm head-PCX-EGFP co-injection, but not after injection of pCX-EGFP DNA alone (see Example 2; Table 1). The frequency of such ±mosaics implies that tg DNA integration was sometimes delayed until after the first S-phase post-injection. Such delayed integration apparently did not occur unless tg DNA had been co-injected with a sperm head. One interpretation of this is that sperm-derived material stabilizes exogenous DNA within the early embryo, thereby facilitating delayed integration; in the absence of such material (in parthenogenotes, for example) the exogenous DNA would be degraded before it could integrate.

We claim:

1. A method for obtaining a transgenic embryo, comprising the steps of:
incubating a nucleic acid that is exogenous to the embryo with a membrane-disrupted sperm head or a demembranated sperm head for a period of time;
co-inserting the exogenous nucleic acid and sperm head into an unfertilized oocyte to form a transgenic fertilized oocyte; and
allowing the transgenic fertilized oocyte to develop into a transgenic embryo.

2. The method of claim 1, wherein the co-inserting step is accomplished by piezo-electrically actuated microinjection.

3. The method of claim 2, wherein the exogenous nucleic acid and the membrane-disrupted sperm head are co-inserted into the cytoplasm of the unfertilized oocyte.

4. The method of claim 1, wherein the membrane-disrupted sperm head is obtained from a spermatozoon that has been frozen and thawed.

5. The method of claim 1, wherein the membrane-disrupted sperm head is obtained from a rehydrated freeze-dried spermatozoon.

6. The method of claim 1, wherein the sperm head is a demembranated head comprising the nucleus and perinuclear materials.

7. The method of claim 6, wherein the membrane-disrupted sperm head is obtained from a detergent-treated spermatozoon.

8. The method of claim 1, wherein the unfertilized oocyte is a metaphase II oocyte.

9. The method of claim 1, wherein the incubating time period is about 30 seconds to, about 5 minutes.

10. The method of claim 9, wherein the incubating time period is about 45 seconds to about 3 minutes.

11. The method of claim 10, wherein the incubating time period is about 1 minute to about 2 minutes.

12. The method of claim 1, wherein the exogenous nucleic acid comprises more than one transgene.

13. The method of claim 1, further comprising the step of allowing the transgenic embryo to develop into a live offspring.

14. The method of claim 13, wherein the allowing step comprises the substep of transplanting the transgenic embryo into a surrogate mother.

15. The method of claim 14, wherein the mammal is selected from the group consisting of primates, ovines, bovines, porcines, ursines, felines, canines, equines and rodents.

16. The method of claim 1, wherein the oocyte and the sperm head are from a mammal.

17. The method of claim 1, wherein the oocyte and the sperm head are from an invertebrate.

18. The method of claim 1, wherein the oocyte and the sperm head are from a fish, an amphibian, a reptile or a bird.

19. The method of claim 1, wherein the oocyte and the sperm head are from a sea urchin, a lobster, an abalone, or a shellfish.

20. A method for obtaining a transgenic embryo, comprising the steps of:
obtaining a membrane-disrupted sperm head or a demembranated sperm head;
mixing the membrane-disrupted sperm head or demembranated sperm head with a nucleic acid that is exogenous to the embryo;
co-inserting the mixture into an isolated unfertilized metaphase II oocyte to form a transgenic fertilized oocyte; and
allowing the transgenic fertilized oocyte to develop into a transgenic embryo.
